(19) United States
(12) Patent Application Publication
(10) Pub. No.: US 2002/0128478 A1
(43) Pub. Date: Sep. 12, 2002

(54) Process for a carbon-carbon coupling reaction of aryl halides with olefins by heterogeneous catalysts

(76) Inventors: Shane W. Krska, New Providence, NJ (US); Thorsten S. Rosner, North Plainfield, NJ (US); Yongkui Sun, Bridgewater, NJ (US)

Correspondence Address: MERCK AND CO INC, P O BOX 2000, RAHWAY, NJ, 070650907

(21) Appl. No.: 10/090,178

(22) Filed: Mar. 4, 2002

Related U.S. Application Data
(60) Provisional application No. 60/272,733, filed on Mar. 2, 2001.

(51) Int. Cl. C07D 279/12; C07D 277/04

(52) U.S. Cl. 544/59; 544/224; 544/298; 544/322; 544/408; 546/290; 546/304; 548/182; 548/225; 548/316.4; 548/557; 564/194; 564/426; 568/807; 570/200

(57) ABSTRACT

This invention relates to a process for the Heck coupling reaction where heterogeneous palladium catalysts are used to activate aryl halides for a carbon-carbon coupling with olefins in the presence a base and an aprotic solvent to produce aryl-olefin compounds. The process, in particular, provides for the use of aryl chlorides substituted with electron-withdrawing or electron-donating group for the cross coupling with olefins.

### FIELD OF THE INVENTION

The present invention is directed to a process for the activation of an aryl halide for the Heck coupling reaction with an olefin by a heterogeneous catalyst.

### BACKGROUND OF THE INVENTION

Aromatic halides are important starting material in synthetic organic chemistry. Aryl halides can be used for the introduction of the aromatic moiety to various organic compounds, which have application for manufacturing many valuable aromatic products for industrial needs. Poylfunctional derivatives of benzene, naphthalene, and a number of aromatic heterocycles are of special interest for the production of polymers, medicines, dye and agricultural chemicals. However, unlike alkyl halides, aromatic halides exhibit low reactivity toward nucleophiles because of the inertness of their carbon-halogen bond. Enhancement of the poor reactivity of aryl halides thus still remains a challenging problem in the field of synthetic chemistry.

Much attention has been focused for many years on activation, cleavage, and functionalization of the aryl halide carbon-halogen bond. Because of the availability of aryl chlorides in bulk quantities at low cost, much of the recent work has focused on the activation of carbon-chlorine bonds of aryl chlorides using homogeneous catalysis. These reactions involve a carbon-carbon coupling of an aryl halide with an olefin (known as the Heck reaction) with a homogeneous catalyst having phosphine ligands. These phosphine ligands play an important role in the reactivity and selectivity of these reactions. However, the homogeneously catalyzed Heck reactions, particularly those employing phosphine ligands are less suitable for industrial applications. Furthermore, typical homogeneous palladium catalysts such as Pd(dba)₂ or Pd(PPh₃)₄, which are often used in the Heck coupling of aryl bromides, do not work well with aryl chlorides yielding poor results.

Littke et al. (J. Org. Chem., 64,10-11 (1999), “Heck Reaction in the Presence of P(t-Bu)₃”) and Grushin et al. (Chem. Rev., 94, 1047 (1994), “Transformation of Chloroarens, Catalyzed by Transition-Metal Complexes”) report the homogeneous palladium catalyzed Heck reactions of an aryl chloride in the presence of phosphine ligands. These homogeneous catalytic reactions usually require a difficult separation process to obtain the final products involving precipitation and chromatographic techniques, or multi-step extraction process.

In contrast, the Heck coupling reaction using a heterogeneous catalyst with an aryl chloride without the ligands is desirable because the heterogeneous catalysis provides advantages for large-scale synthesis with industrial applications. Some of the advantages are ease of product separation and recycling of the catalysts, which provide an overall simplified and cost-effective process. In spite of the industrial applicability and process advantages, only a few examples of heterogeneously catalyzed Heck reactions are known today, particularly those involving an aryl chloride. In most reported cases, the reactions involve utilization of aryl bromide, harsh reaction conditions, or catalysts that are not commercially available (e.g. colloidal Pd or Pd-TMS11 catalysts).

Eisenstadt et al. (U.S. Pat. No. 5,187,303) is directed to a process for the preparation of octyl methoxycinnamate by reacting aryl bromide with octyl acrylate in the presence of a base and a coupling catalyst.

Eisenstadt (Chemical Industries, “Catalysis of Organic Reactions”, Decker, N.Y., 1998, 75, 415) relates to a heterogeneous Pd/C catalyzed Heck reaction using aryl bromides.

Mehnert et al. (J. Am. Chem. Soc. 120, 12289-12296 (1998), “Heterogeneous Heck Catalysis with Palladium-Grafted Molecular Sieves”) relates to carbon-carbon coupling reactions with activated and non-activated aryl substrates by palladium-grafted mesoporous molecular sieves, designated Pd-TMS11.

In view of the above, an object of the present invention is to develop a process for activating an aryl halide for the Heck coupling reaction with an olefin using a commercially available heterogeneous catalyst to prepare an aryl-olefin compound for wide industrial applications.

### SUMMARY OF THE INVENTION

The present invention relates to a process for a carbon-carbon coupling reaction by heterogeneous catalysis to activate aryl halides or heteroaryl halides, more specifically aryl chloride, for cross coupling with olefins to produce aryl-olefin compounds.

The present invention provides a process for preparing a compound of Formula I,

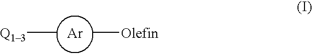

comprising: p1 reacting

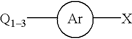

with an olefin, wherein

Ar is:

(1) aryl, wherein aryl is defined as phenyl, naphthyl, anthracenyl, or phenanthrenyl substituted with one to three substituents of Q, or

(2) heteroaryl, wherein heteroaryl is defined as a monocyclic or bicyclic aromatic ring of 5 to 10 carbon atoms containing from 1 to 3 heteroatoms selected from O, N or S, and the heteroaryl being substituted with one to three substituents of Q;

Q is:

(1) halo, wherein halo is fluoro or chloro,

(2) (C₁—C₆)-alkyl,

(3) (C₁—C₆)-alkoxy,

(4) phenyl,

(5) oxo,

(6) hydroxy,

(7) NHCO(C —C₆—C₆)-alkyl,

(8) NR⁴R⁵, wherein R⁴ and R⁵ are independently hydrogen or (C₁—C₆)-alkyl,

(9) nitro,

(10) CO₂H,

(11) (C₁—C₄)-perfluoroalkyl,

(12) (C₁—C₄)-perfluoroalkoxy,

(13) cyano,

(14) SO₃H,

(15) CO₂(C₁—C₆)-alkyl,

(16) CO(C₁—C₆)-alkyl,

(17) CCl₃,

(18) CHO, or

(19) NR₃⁺ wherein R is hydrogen or (C₁—C₆)-alkyl;

X is Cl, Br, or I;

olefin is H(R¹)C=C(R²)( R³), cycloolefin, or heterocyclic olefin, wherein R^{1, R}² and R³are independently:

(1) hydrogen,

(2) (C₁—C₁₂)-alkyl,

(3) (C₂—C₁₂)-alkenyl,

(4) (C₁—C₁₂)-alkoxy,

(5) heteroaryl, wherein heteroaryl is defined as above,

(6) aryl, wherein aryl is defined as above,

(7) nitrile,

(8) silyl,

(9) NR⁴R⁵, wherein R⁴ and R⁵ are as defined above,

(10) NH(C₁—C₆)-alkyl-NR⁴R⁵, wherein R⁴ and R⁵ are as defined above,

(11) halo, wherein halo is fluoro, chloro, bromo, or iodo,

(12) (C₁—C ₂)-alcohol,

(13) CO₂H,

(14) CO₂(C₁—C₁₂)-alkyl, or

(15) SR, wherein R is hydrogen or (C₁—C₆)-alkyl;

cycloolefin is defined as a monocyclic or bicyclic ring of 5 to 10 carbon atoms containing at least one unsaturation at any point in the ring, and the cycloolefin being optionally substituted with one to three substituents as defined above in R¹, R² and R³; and

heterocyclic olefin is defined as a monocyclic or bicyclic ring of a 5 to 10 carbon atoms with at least one unsaturation at any point in the ring containing from 1 to 3 heteroatoms selected from the group consisting of O, N, and S and the heterocyclic olefin being optionally substituted with one to three substituents as defined above in R¹, R² and R³;

in the presence of a heterogeneous catalyst and a base at a temperature range of about 100° C. to about 180° C. in an aprotic solvent to generate the compound of Formula I.

### DETAILED DESCRIPTION OF THE INVENTION

The present invention is directed to a process for a carbon-carbon coupling reaction between aryl halide or heteroaryl halide, which is substituted with either electron donating or electron withdrawing group, and olefin using heterogeneous palladium (Pd) catalyst to produce the corresponding aryl-olefin or heteroaryl-olefin compounds.

An embodiment of the present invention is a process for preparing a compound of Formula I,

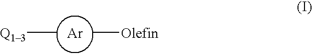

comprising:

reacting

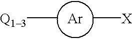

with an olefin, wherein

Ar is:

(1) aryl, wherein aryl is defined as phenyl, naphthyl, anthracenyl, or phenanthrenyl substituted with one to three substituents of Q, or

(2) heteroaryl, wherein heteroaryl is defined as a monocyclic or bicyclic aromatic ring of 5 to 10 carbon atoms containing from 1 to 3 heteroatoms selected from O, N or S, and the heteroaryl being substituted with one to three substituents of Q;

Q is:

(1) halo, wherein halo is fluoro or chloro,

(2) (C₁—C₆)-alkyl,

(3) (C₁—C₆)-alkoxy,

(4) phenyl,

(5) oxo,

(6) hydroxy,

(7) NHCO(C—C₆)-alkyl,

(8) NR⁴R⁵, wherein R⁴ and R⁵ are independently hydrogen or (C₁—C₆)-alkyl,

(9) nitro,

(10) CO₂H,

(11) (C₁—C₄)- perfluoroalkyl,

(12) (C₁—C₄)- perfluoroalkoxy,

(13) cyano,

(14) SO₃H,

(15) CO₂(C₁—C₆)-alkyl,

(16) CO(C₁—C₆)-alkyl,

(17) CCl₃,

(18) CHO, or

(19) NR₃⁺wherein R is hydrogen or (C₁—C₆)-alkyl;

X is Cl, Br, or I;

olefin is H(R¹)C=C(R²)(R³), cycloolefin, or heterocyclic olefin, wherein R¹, R² and R³ are independently:

(1) hydrogen,

(2) (C₁—C₁₂)-alkyl,

(3) (C₂—C₁₂)-alkenyl,

(4) (C₁—C ₁₂)-alkoxy,

(5) heteroaryl, wherein heteroaryl is defined as above,

(6) aryl, wherein aryl is defined as above,

(7) nitrile,

(8) silyl,

(9) NR⁴R⁵, wherein R⁴ and R⁵ are as defined above,

(10) NH(C₁—C₆)-alkyl-NR⁴R⁵, wherein R⁴ and R⁵ are as defined above,

(11) halo, wherein halo is fluoro, chloro, bromo, or iodo,

(12) (C₁—C ₂)-alcohol,

(13) CO₂H,

(14) CO₂(C₁—C₁₂)-alkyl, or

(15) SR, wherein R is hydrogen or (C₁—C₆)-alkyl;

cycloolefin is defined as a monocyclic or bicyclic ring of 5 to 10 carbon atoms containing at least one unsaturation at any point in the ring, and the cycloolefin being optionally substituted with one to three substituents as defined above in R¹, R² and R³; and

heterocyclic olefin is defined as a monocyclic or bicyclic ring of a 5 to 10 carbon atoms with at least one unsaturation at any point in the ring containing from 1 to 3 heteroatoms selected from the group consisting of O, N, and S and the heterocyclic olefin being optionally substituted with one to three substituents as defined above in R¹, R² and R³; in the presence of a heterogeneous catalyst and a base at a temperature range of about 100° C. to about 180° C. in an aprotic solvent to generate the compound of Formula I.

A preferred embodiment of the present invention is a process for preparing a compound of Formula Ia,

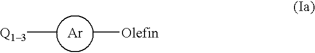

comprising:

reacting

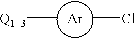

with an olefin, wherein

Ar is:

(1) aryl, wherein aryl is defined as phenyl, naphthyl, anthracenyl, or phenanthrenyl substituted with one to three substituents of Q, or

(2) heteroaryl, wherein heteroaryl is defined as a monocyclic or bicyclic aromatic ring of 5 to 10 carbon atoms containing from 1 to 3 heteroatoms selected from O, N or S, and the heteroaryl being substituted with one to three substituents of Q;

Q is:

(1) halo, wherein halo is fluoro or chloro,

(2) (C_{1—C}₆)-alkyl,

(3) (C₁—C₆)-alkoxy,

(4) phenyl,

(5) oxo,

(6) hydroxy,

(7) NHCO(C₁—C₆)-alkyl,

(8) NR⁴R⁵, wherein R⁴ and R⁵ are independently hydrogen or (C₁—C₆)-alkyl,

(9) nitro,

(10) CO₂H,

(11) (C₁—C₄)- perfluoroalkyl,

(12) (C₁—C₄)- perfluoroalkoxy,

(13) cyano,

(14) SO₃H,

(15) CO₂(C₁—C₆)-alkyl,

(16) CO(C₁—C₆)-alkyl,

(17) CCl₃,

(18) CHO, or

(19) NR₃⁺wherein R is hydrogen or (C₁—C₆)-alkyl;

olefin is H(R¹)C=C(R²)( R³), cycloolefin, or heterocyclic olefin, wherein R¹, R² and R³ are independently:

(1) hydrogen,

(2) (C₁—C₁₂)-alkyl,

(3) (C₂—C₁₂)-alkenyl,

(4) (C₁—C₁₂)-alkoxy,

(5) heteroaryl, wherein heteroaryl is defined as above,

(6) aryl, wherein aryl is defined as above,

(7) nitrile,

(8) silyl,

(9) NR⁴R⁵, wherein R⁴ and R⁵ are as defined above,

(10) NH(C₁—C₆)-alkyl-NR⁴R⁵, wherein R⁴ and R⁵ are as defined above,

(11) halo, wherein halo is fluoro, chloro, bromo, or iodo,

(12) (C₁—C₁₂)-alcohol,

(13) CO₂H,

(14) CO₂(C₁—C₁₂)-alkyl, or

(15) SR, wherein R is hydrogen or (C₁—C₆)-alkyl;

cycloolefin is defined as a monocyclic or bicyclic ring of 5 to 10 carbon atoms containing at least one unsaturation at any point in the ring, and the cycloolefin being optionally substituted with one to three substituents as defined above in R¹R² and R³; and

heterocyclic olefin is defined as a monocyclic or bicyclic ring of a 5 to 10 carbon atoms with at least one unsaturation at any point in the ring containing from 1 to 3 heteroatoms selected from the group consisting of O, N, and S and the heterocyclic olefin being optionally substituted with one to three substituents as defined above in R¹, R² and R³; in the presence of a heterogeneous catalyst and a base at a temperature range of about 100° C. to about 180° C. in an aprotic solvent to generate the compound of Formula Ia.

Another preferred embodiment of the present invention is a process for preparing a compound of Formula 1b,

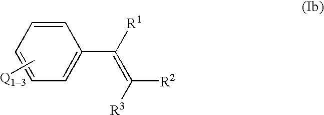

comprising:

reacting an aryl chloride of structural formula

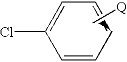

with an olefin of formula H(R¹)C=C(R²)(R³), wherein

Q is:

(1) halo, wherein halo is fluoro or chloro,

(2) (C₁—C₆)-alkyl,

(3) (C₁—C₆)-alkoxy,

(4) phenyl,

(5) oxo,

(6) hydroxy,

(7) NHCO(C₁—C₆)-alkyl,

(8) NR⁴R⁵, wherein R⁴ and R⁵ are independently hydrogen or (C₁—C₆)-alkyl,

(9) nitro,

(10) CO₂H,

(11) (C₁—C₄)- perfluoroalkyl,

(12) (C₁—C₄)- perfluoroalkoxy,

(13) cyano,

(14) SO₃H,

(15) CO₂(C₁—C₆)-alkyl,

(16) CO(C₁—C₆)-alkyl,

(17) CCl_{3,}

(18) CHO, or

(19) NR₃⁺ wherein R is hydrogen or (C₁—C₆)-alkyl; and

R¹R² and R³ are independently:

(1) hydrogen,

(2) (C₁—C₁₂)-alkyl,

(3) (C₂—C₁₂)-alkenyl,

(4) (C₁—C₁₂)-alkoxy,

(5) heteroaryl, wherein heteroaryl is defined as above,

(6) aryl, wherein aryl is defined as above,

(7) nitrile,

(8) silyl,

(9) NR⁴R⁵ wherein R⁴ and R⁵ are as defined above,

(10) NH(C₁—C₆)-alkyl-NR⁴R⁵ wherein R⁴ and R⁵ are as defined above,

(11) halo, wherein halo is fluoro, chloro, bromo, or iodo,

(12) (C₁—C₂)-alcohol,

(13) CO₂H,

(14) CO₂(C₁—C₂)-alkyl, or

(15) SR, wherein R is hydrogen or (C₁—C₆)-alkyl;

in the presence of a heterogeneous catalyst and a base at a temperature range of about 100° C. to about 180° C. in an aprotic solvent to generate the compound of Formula Ib.

The process as recited above, wherein the Q is substituted in the position 4 of the phenyl ring.

The process as recited above, wherein the heterogeneous catalyst is finely dispersed palladium on a solid support.

The process as recited above, wherein the solid support is selected from the group consisting of carbon (Pd/C), silica, alumina, titania, and crystalline mesopourous zeolitic materials.

The process as recited above, wherein the heterogeneous palladium catalyst is finely dispersed palladium without a solid support.

The process as recited above, wherein the finely dispersed palladium is finely dispersed palladium metal (Pd Black) or finely dispersed palladium generated from homogeneous palladium acetate.

The process as recited above, wherein the heterogeneous palladium catalyst is finely dispersed palladium (colloidal) stabilized by organic polymers.

The process as recited above, wherein the aprotic solvent selected from the group consisting of N,N-dimethylacetamide (DMA), dioxane, N,N-dimethylformamide (DMF), N-methylpyrrolidinone (NMP), dioxane, ethylene glycol dimethyl ether (DME), diethoxymethane (DEM), tetrahydrofuran (THF), water, and a mixture thereof. A preferred aprotic solvent is DMA, 1,4-dioxane, or a mixture thereof. A more preferred aprotic solvent is the mixture of DMA and 1,4-dioxane at a ratio of about 1:1 to about 1:5, preferably about 1:3.

The process as recited above, wherein the base is selected from the group consisting of sodium acetate, potassium acetate, cesium acetate, triethylamine, trimethylamine, ethyldimethylamine, tri-n-propylamine, 1,4-diazabicyclio[2.2.2]octane, 1,8-diazabicyclo[5.4.0.]undec-7-ene (DBU), pyridine, lutidine, collidine, 4-dimethylaminomethyl-pyridine, sodium carbonate, potassium carbonate, cesium carbonate, sodium bicarbonate, potassium bicarbonate potassium, potassium methoxide, potassium ethoxide, sodium methoxide, sodium ethoxide, sodium tartrate, potassium tartrate, potassium bitartrate, sodium tartrate, and sodium bitartrate. The preferred base is sodium acetate, potassium acetate, cesium acetate. The base may be present in an amount of about one to five equivalents, preferably about one to two equivalents, relative to the amount of aryl halide.

The process as recited above, wherein the temperature range is about 140° C. to about 160° C.

The process as recited above further comprises a radical scavenger selected from the group consisting of 4-methoxyphenol, bis (tert-butyl)hydroxy toluene (BHT), 1,4-benzoquinone, and 4-tert-butylcatechol.

It is further understood that the substituents recited above would include the definitions recited below, and unless otherwise stated or indicated, the definitions shall apply throughout the specification and claims.

As used herein, the term “alkyl,” unless otherwise indicated, includes those alkyl groups of a designated number of carbon atoms of either a straight, branched, or cyclic configuration. Examples of “alkyl” include, but are not limited to: methyl, ethyl, propyl, isopropyl, butyl, sec-butyl, tert-butyl, pentyl, hexyl, heptyl, and the like. Examples of cycloalkyls are cyclopropyl, cyclobutyl, cyclopentyl, cyclohexyl, cycloheptyl, norbornyl, 1,1,3,3-tetramethyl butyl, and the like.

The term “alkoxy” represents an alkyl group of indicated number of carbon atoms attached through an oxygen bridge, such as methoxy, ethoxy, propoxy, isopropoxy, butoxy, tert-butoxy, pentoxy, and the like.

The term “olefin” as used herein includes alkene, cycloolefin, heterocyclic olefin. Alkene refers to hydrocarbon of a specific number of carbon atoms of either a straight or branched configuration having at least one unsaturation that may occur at any point along the chain. Alkene may be optionally substituted with one to three substituents as set forth in the embodiment recited above. Examples of alkene include, but are not limited to: ethene, propene, butene, pentene, cyclohexene and the like. Examples of substituted alkene include, but are not limited to: alkylacrylate such as butylacrylate and allylic alcohol.

The term cycloolefin refers to a monocyclic or bicyclic ring of 5 to 10 carbon atoms containing at least one unsaturation at any point in the ring. The cycloolefin may be optionally substituted with one to three substituents as set forth in the embodiment recited above. Examples of cycloolefin are, but are not limited to: cyclopropene, cyclobutene, cyclopentene, cyclohexene, 2-cyclohexene-1-one, cyclooctene, cyclobutadiene, cyclopentadiene, cyclohexadiene, cycloheptatriene, and the like.

The term heterocyclic olefin as used herein refers to a monocyclic or bicyclic ring of a 5 to 10 carbon atoms with at least one unsaturation at any point in the ring containing from 1 to 3 heteroatoms selected from the group consisting of O, N, and S. The heterocyclic olefin may be optionally substituted with one to three substituents as set forth in the embodiment recited above. Examples of the heterocyclic olefin is, but are not limited to: 2,3-dihydrofuran, 2,3-dihydrothiopehen, 2,3-dihydropyrrole, 2,3-dihydropyran, 5,6-dihydro-4methoxy-2H-pyran, and the like.

The term “aryl,” is defined as phenyl, naphthyl, anthracenyl, phenanthrenyl and the like which is substituted with one to three substituents as set forth in the embodiments recited above.

The term “heteroaryl” as utilized herein, unless specifically defined otherwise, is intended to include a monocyclic or bicyclic (fused) aromatic ring of 5 to 10 carbon atoms containing from 1 to 3 heteroatoms selected from O, N and/or S, and the heterocyclyl being substituted with one to three substituents as set forth in the embodiments recited above. Heteroaryl groups within the scope of this definition include, but are not limited to: acridinyl, carbazolyl, cinnolinyl, quinoxalinyl, pyrrazolyl, indolyl, benzotriazolyl, furanyl, thienyl, thiazoyl, benzothienyl, benzofuranyl, quinolinyl, isoquinolinyl, pyrazinyl, pyridazinyl, pyridinyl, pyrimidinyl, and pyrrolyl.

Halogen, halide and halo as used herein refer to bromine, chlorine, fluorine and iodine.

The term “radical scavenger” as used herein refers to an inhibitor of free radicals, which are known to be formed by ionizing radiation having an unpaired electron and act as initiators or reactive intermediates in oxidation, combustion, photolysis and polymerization.

The process of the present invention is illustrated by the following reaction scheme:

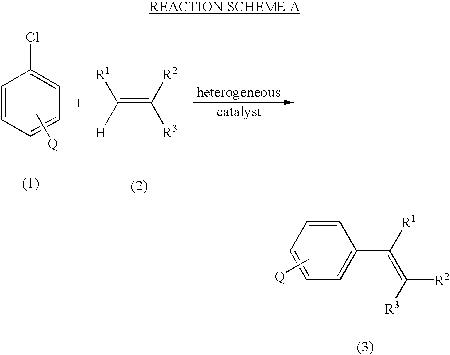

In Reaction Scheme A, the carbon-carbon coupling reaction can be accomplished by reacting an aryl chloride (1), which is substituted with either electron-donating or electron-withdrawing group (denoted as Q), with an olefin (2) in the presence of a palladium heterogeneous catalyst and a base in an aprotic solvent to produce an aryl-olefin compound (3). More than one electron-withdrawing or electron- donating group can be substituted in the aryl ring, and at least one electron-withdrawing group may be present depending on the number of electron-donating group in the aryl ring. Additionally, electron-withdrawing or electron-donating group can be substituted anywhere in the aryl ring such as ortho, meta or para position. Examples of electron- withdrawing or electron-donating group are, but not limited to: F, Cl, Br, I, CH₃, C₂H₅, OCH₃, NHCOCH₃, C₆H₅, CN, SO₃H, CO₂H, CHO, NO₂, CF₃, NR₃⁺ and CCl_{3.}

The coupling reaction can be carried out at a temperature range of about 100° C. to about 180° C., preferably about 100° C. to 160° C. and more preferably about 140° C. to 160° C., using an aprotic solvent such as DMA (N,N-dimethylacetamide), DMF (N,N-dimethylformamide), dioxane and water. The reaction in the solvent mixture of DMA and 1,4-dioxane (ratio 3:1) at a temperature about 140° C. is preferred. Addition of a base is required and the suitable base can be selected from: sodium acetate, potassium acetate, cesium acetate, triethylamine, trimethylamine, ethyldimethylamine, tri-n-propylamine, 1,4-diazabicyclio[2.2.2]octane, 1,8-diazabicyclo[5.4.0.]undec-7-ene (DBU), pyridine, lutidine, collidine, 4-dimethylaminomethyl-pyridine, sodium carbonate, potassium carbonate, cesium carbonate, sodium bicarbonate, potassium bicarbonate potassium, potassium methoxide, potassium ethoxide, sodium methoxide, sodium ethoxide, sodium tartrate, potassium tartrate, potassium bitartrate, sodium tartrate, and sodium bitartrate. The preferred base is sodium acetate, potassium acetate, and cesium acetate. The base may be added in an amount of about one to five equivalents, preferably about one to two equivalents relative to the amount of aryl chloride.

A radical scavenger may be added to prevent or minimize the polymerization of the olefin during the reaction. The radical scavenger can be present in an amount of about 0.1 to about 5 equivalents, preferably about 0.1 to about 2 equivalents relative to the amount of aryl chloride. Examples of suitable radical scavenger are 4-methoxyphenol, bis(tert-butyl)hydroxy toluene (BHT), 1,4-benzoquinone, and 4-tert-butylcatechol.

Heterogeneous catalysts are those which contain a palladium (Pd) source including those that are finely dispersed palladium with or without a solid support; or finely dispersed palladium stabilized by organic polymers such as poly(vinylpyrrolidinones), poly(vinyl alcohol) and poly (methyl vinyl ether). The preferable palladium catalyst system is one with an optimal palladium level of about 0.1 mole % to about 15 mole%, preferably about 3 mole% to about 10 mole%.

Finely dispersed palladium on a solid support includes palladium supported on carbon (Pd/C), silica, alumina, titania, and mesopourous or zeolitic materials. The state of the palladium can be in a reduced or non-reduced form in which case it can be reduced in situ with any suitable reducing agents such as aryl boronic acids, potassium or sodium formate, hydrogen, borohydride reagents, silanes, aluminum hydride reagents, hydrazine and the like.

Finely dispersed palladium without a solid support includes finely dispersed palladium metal (Pd Black) and finely dispersed palladium generated from homogeneous palladium sources (such as palladium acetate) by action of a suitable reducing agent such as potassium or sodium formate, hydrogen, borohydride reagents, silanes, aluminum hydride reagents, hydrazine and the like. Finely dispersed palladium stabilized by organic polymers includes colloidal palladium stabilized by organic polymers such as poly(vinylpyrrolidinones), poly(vinyl alcohol) and poly(methyl vinyl ether).

The following examples illustrate the process of the present invention, and as such are not to be considered as limiting the invention set forth in the claims appends hereto.

### EXAMPLE 1

Heck Reaction of 4—Chlorotrifluorotoluene with Butylacrylate

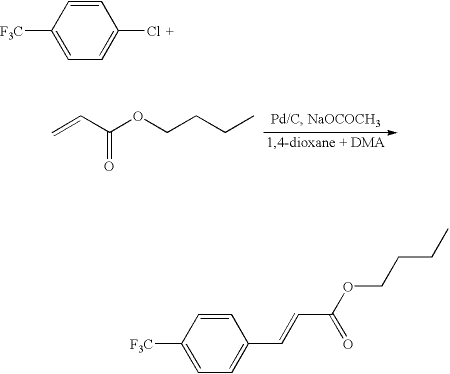

Reagents of 4-chlorotrifluorotoluene (0.82 g), butylacrylate (0.78 g), sodium acetate (0.60 g), 1,4-dioxane (0.80 g), DMA (2.1 g), palladium catalyst (5% Pd/C, 0.40 g) and 4-methoxyphenol (0.61 g) are measured gravimetrically and filled into the Schlenck tube. The tube is sealed and then evacuated and refilled with nitrogen three times. The oil bath is preheated to about 160° C. The Schlenck tube is placed in the oil bath and the reaction is carried out over night. The solution is then filtered, and about 0.5 ml of the solution is diluted with acetonitrile to about 100 ml for a HPLC analysis.

HPLC analysis: UV detection at 220 nm; Column: Inertsil 5u ODS3; Flow: 1.5 ml/min.; Solvent A: acetonitrile; Solvent B: water (0.1% buffer, H₃PO₄); Retention time (min): butylacrylate (14.8), 4-chlorotrifluorotoluene (18.4), aryl-olefin product (21.6).

### EXAMPLE 2

Heck-Reaction of 4—Chlorotoluene with Butylacrylate

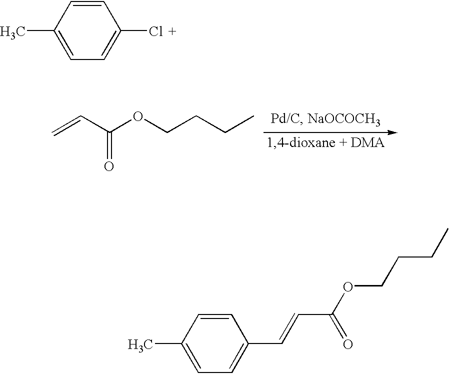

Reagents of 4-chlorotoluene (0.53 g), butylacrylate (0.78 g), sodium acetate (0.62 g), 1,4-dioxane (1.77 g), DMA (1.12 g), palladium catalyst (5% Pd/C, 0.40 g) and 4-methoxyphenol (0.61 g) are measured gravimetrically and filled into the Schlenck tube. The tube is sealed and then evacuated and refilled with nitrogen three times. The oil bath is preheated to about 160° C. The Schlenck tube is placed in the oil bath and the reaction is carried out over night. The solution is then filtered, and about 0.5 ml of the solution is diluted with acetonitrile to about 100 ml for a HPLC analysis.

HPLC analysis: UV detection at 220 nm; Column: Inertsil 5u ODS3; Flow: 1.5 ml/min.; Solvent A: acetonitrile; Solvent B: water (0.1% buffer, H₃PO₄); Retention time (min): butylacrylate (14.8), 4-chlorotoluene (18.1), aryl-olefin product (21.5).

### EXAMPLE 3

Heck-Reaction of 4—Chloroanisol with Butylacrylate

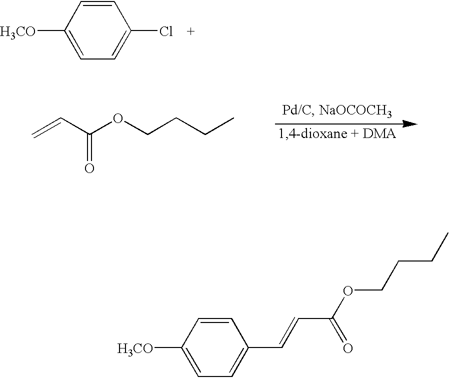

Reagents of 4-chloroanisol (0.59 g), butylacrylate (0.78 g), sodium acetate (0.62 g), 1,4-dioxane (1.84 g), DMA (1.13 g), palladium catalyst (5% Pd/C, 0.41 g) and 4-methoxyphenol (0.59 g) are measured gravimetrically and filled into the Schlenck tube. The tube is sealed and then evacuated and refilled with nitrogen three times. The oil bath is preheated to about 160° C. The Schlenck tube is placed in the oil bath and the reaction is carried out over night. The solution is then filtered, and about 0.5 ml of the solution is diluted with acetonitrile to about 100ml for a HPLC analysis.

HPLC analysis: UV detection at 220 nm; Column: Inertsil 5u ODS3; Flow: 1.5 ml/min.; Solvent A: acetonitrile; Solvent B: water (0.1% buffer, H₃PO₄); Retention time (min): butylacrylate (14.8), 4-chloroanisol (16.1), aryl-olefin product (19.7).

What is claimed is:

1. A process for preparing a compound of Formula I, 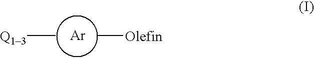
comprising:
reacting 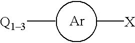
with an olefin, wherein
Ar is:
(1) aryl, wherein aryl is defined as phenyl, naphthyl, anthracenyl, or phenanthrenyl substituted with one to three substituents of Q, or
(2) heteroaryl, wherein heteroaryl is defined as a monocyclic or bicyclic aromatic ring of 5 to 10 carbon atoms containing from 1 to 3 heteroatoms selected from O, N or S, and the heteroaryl being substituted with one to three substituents of Q;
Q is:
(1) halo, wherein halo is fluoro or chloro,
(2) (C₁—C₆)-alkyl,
(3) (C₁—C₆)-alkoxy,
(4) phenyl,
(5) oxo,
(6) hydroxy,
(7) NHCO(C₁—C₆)-alkyl,
(8) NR⁴R⁵, wherein R⁴ and R⁵ are independently hydrogen or (C₁—C₆)-alkyl,
(9) nitro,
(10) CO₂H,
(11) (C₁—C₄)- perfluoroalkyl,
(12) (C—C₄)- perfluoroalkoxy,
(13) cyano,
(14) SO₃H,
(15) CO₂(C₁—C₆)-alkyl,
(16) CO(C₁—C₆)-alkyl,
(17) CCl₃,
(18) CHO, or
(19) NR₃⁺ wherein R is hydrogen or (C₁—C₆)-alkyl;
X is Cl, Br, or I;
olefin is H(R¹)C=C(R²)( R³), cycloolefin, or heterocyclic olefin, wherein R¹, R²and R³are independently:
(1) hydrogen,
(2) (C₁—C ₁₂)-alkyl,
(3) (C₂—C₁₂)-alkenyl,
(4) (C₁—C₁₂)-alkoxy,
(5) heteroaryl, wherein heteroaryl is defined as above,
(6) aryl, wherein aryl is defined as above,
(7) nitrile,
(8) silyl,
(9) NR⁴R⁵, wherein R⁴ and R⁵ are as defined above,
(10) NH(C₁—C₆)-alkyl-NR⁴R⁵, wherein R⁴ and R⁵ are as defined above,
(11) halo, wherein halo is fluoro, chloro, bromo, or iodo,
(12) (C₁—C₁₂)-alcohol,
(13) CO₂H,
(14) CO₂(C₁—C₁₂)-alkyl, or
(15) SR, wherein R is hydrogen or (C₁—C₆)-alkyl;
cycloolefin is defined as a monocyclic or bicyclic ring of 5 to 10 carbon atoms containing at least one unsaturation at any point in the ring, and the cycloolefin being optionally substituted with one to three substituents as defined above in R¹, R² and R³; and
heterocyclic olefin is defined as a monocyclic or bicyclic ring of a 5 to 10 carbon atoms with at least one unsaturation at any point in the ring containing from 1 to 3 heteroatoms selected from the group consisting of O, N, and S and the heterocyclic olefin being optionally substituted with one to three substituents as defined above in R¹, R² and R³;
in the presence of a heterogeneous catalyst and a base at a temperature range of about 100° C. to about 180° C. in an aprotic solvent to generate the compound of Formula I.

2. The process of claim 1, wherein the heterogeneous catalyst is finely dispersed palladium on a solid support.

3. The process of claim 2, wherein the solid support is selected from the group consisting of carbon (Pd/C), silica, alumina, titania, and crystalline mesopourous zeolitic materials.

4. The process of claim 3, wherein the heterogeneous palladium catalyst is finely dispersed palladium without a solid support.

5. The process of claim 4, wherein the finely dispersed palladium is finely dispersed palladium metal (Pd Black) or finely dispersed palladium generated from homogeneous palladium acetate.

6. The process of claim 5, wherein the heterogeneous palladium catalyst is finely dispersed palladium (colloidal) stabilized by organic polymers.

7. The process of claim 6, wherein the aprotic solvent is selected from the group consisting of N,N-dimethylacetamide (DMA), dioxane, N,N-dimethylformamide (DMF), N-methylpyrrolidinone (NMP), dioxane, ethylene glycol dimethyl ether (DME), diethoxymethane (DEM), tetrahydrofuran (THF), water, and a mixture thereof.

8. The process of Claim 7, wherein the aprotic solvent is DMA, 1,4-dioxane, or a mixture thereof.

9. The process of claim 8, wherein the aprotic solvent is the mixture of DMA and 1,4-dioxane at a ratio of about 1:1 to about 1:5.

10. The process of claim 9, wherein the ratio of DMA to 1,4-dioxane is about 1:3.

11. The process of claim 10, wherein the base is selected from the group consisting of sodium acetate, potassium acetate, cesium acetate, triethylamine, trimethylamine, ethyldimethylamine, tri-n-propylamine, 1,4-diazabicyclio[2.2.2]octane, 1,8-diazabicyclo[5.4.0.]undec-7-ene (DBU), pyridine, lutidine, collidine, 4-dimethylaminomethyl-pyridine, sodium carbonate, potassium carbonate, cesium carbonate, sodium bicarbonate, potassium bicarbonate potassium, potassium methoxide, potassium ethoxide, sodium methoxide, sodium ethoxide, sodium tartrate, potassium tartrate, potassium bitartrate, sodium tartrate, and sodium bitartrate.

12. The process of claim 11, wherein the temperature range is about 140° C. to about 160° C.

13. The process of claim 1, which further comprises a radical scavenger selected from the group consisting of 4-methoxyphenol, bis(tert-butyl)hydroxy toluene (BHT), 1,4-benzoquinone, and 4-tert-butylcatechol.

14. A process for preparing a compound of Formula Ia, 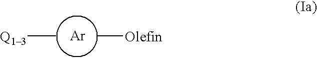
comprising:
reacting 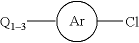
with an olefin, wherein
Ar is:
(1) aryl, wherein aryl is defined as phenyl, naphthyl, anthracenyl, or phenanthrenyl substituted with one to three substituents of Q, or
(2) heteroaryl, wherein heteroaryl is defined as a monocyclic or bicyclic aromatic ring of 5 to 10 carbon atoms containing from 1 to 3 heteroatoms selected from O, N or S, and the heteroaryl being substituted with one to three substituents of Q;
Q is:
(1) halo, wherein halo is fluoro or chloro,
(2) (C₁—C₆)-alkyl,
(3) (C₁—C₆)-alkoxy,
(4) phenyl,
(5) oxo,
(6) hydroxy,
(7) NHCO(C₁—C₆)-alkyl,
(8) NR⁴R⁵, wherein R⁴ and R⁵ are independently hydrogen or (C₁—C₆)-alkyl,
(9) nitro,
(10) CO₂H,
(11) (C₁—C₄)- perfluoroalkyl,
(12) (C₁—C₄)- perfluoroalkoxy,
(13) cyano,
(14) SO₃H,
(15) CO₂(C₁—C₆)-alkyl,
(16) CO(C₁—C₆)-alkyl,
(17) CCl₃,
(18) CHO, or
(19) NR₃⁺ wherein R is hydrogen or (C₁—C₆)-alkyl;
olefin is H(R¹)C=C(R²)( R³), cycloolefin, or heterocyclic olefin, wherein R¹, R² and R³ are independently:
(1) hydrogen,
(2) (C₁—C ₁₂)-alkyl,
(3) (C₂—C₁₂)-alkenyl,
(4) (C₁—C₁₂)-alkoxy,
(5) heteroaryl, wherein heteroaryl is defined as above,
(6) aryl, wherein aryl is defined as above,
(7) nitrile,
(8) silyl,
(9) NR⁴R⁵, wherein R⁴ and R⁵ are as defined above,
(10) NH(C₁—C₆)-alkyl-NR⁴R⁵, wherein R⁴ and R⁵ are as defined above,
(11) halo, wherein halo is fluoro, chloro, bromo, or iodo,
(12) (C₁—C₁₂)-alcohol,
(13) CO₂H,
(14) CO₂(C₁—C₁₂)-alkyl, or
(15) SR, wherein R is hydrogen or (C₁—C₆)-alkyl;
cycloolefin is defined as a monocyclic or bicyclic ring of 5 to 10 carbon atoms containing at least one unsaturation at any point in the ring, and the cycloolefin being optionally substituted with one to three substituents as defined above in R¹, R² and R³; and
heterocyclic olefin is defined as a monocyclic or bicyclic ring of a 5 to 10 carbon atoms with at least one unsaturation at any point in the ring containing from 1 to 3 heteroatoms selected from the group consisting of O, N, and S and the heterocyclic olefin being optionally substituted with one to three substituents as defined above in R¹, R² and R³;
in the presence of a heterogeneous catalyst and a base at a temperature range of about 100° C. to about 180° C. in an aprotic solvent to generate the compound of Formula Ia.

15. The process of claim 14, wherein the heterogeneous catalyst is finely dispersed palladium on a solid support.

16. The process of claim 15, wherein the solid support is selected from the group consisting of carbon (Pd/C), silica, alumina, titania, and crystalline mesopourous zeolitic materials.

17. The process of claim 16, wherein the heterogeneous palladium catalyst is finely dispersed palladium without a solid support.

18. The process of claim 17, wherein the finely dispersed palladium is finely dispersed palladium metal (Pd Black) or finely dispersed palladium generated from homogeneous palladium acetate.

19. The process of claim 18, wherein the heterogeneous palladium catalyst is finely dispersed palladium (colloidal) stabilized by organic polymers.

20. The process of claim 19, wherein the aprotic solvent selected from the group consisting of N,N-dimethylacetamide (DMA), dioxane, N,N-dimethylformamide (DMF), N-methylpyrrolidinone (NMP), dioxane, ethylene glycol dimethyl ether (DME), diethoxymethane (DEM), tetrahydrofuran (THF), water, and a mixture thereof.

21. The process of claim 20, wherein the aprotic solvent is DMA, 1,4-dioxane, or a mixture thereof.

22. The process of Claim 21, wherein the aprotic solvent is the mixture of DMA and 1,4-dioxane at a ratio of about 1:1 to about 1:5.

23. The process of claim 22, wherein the ratio of DMA to 1,4-dioxane is about 1:3.

24. The process of claim 23, wherein the base is selected from the group consisting of sodium acetate, potassium acetate, cesium acetate, triethylamine, trimethylamine, ethyldimethylamine, tri-n-propylamine, 1,4-diazabicyclio[2.2.2]octane, 1,8-diazabicyclo[5.4.0.]undec-7-ene (DBU), pyridine, lutidine, collidine, 4-dimethylaminomethyl-pyridine, sodium carbonate, potassium carbonate, cesium carbonate, sodium bicarbonate, potassium bicarbonate potassium, potassium methoxide, potassium ethoxide, sodium methoxide, sodium ethoxide, sodium tartrate, potassium tartrate, potassium bitartrate, sodium tartrate, and sodium bitartrate.

25. The process of claim 24, wherein the temperature range is about 140° C. to about 160° C.

26. The process of claim 14, which further comprises a radical scavenger selected from the group consisting of 4-methoxyphenol, bis(tert-butyl)hydroxy toluene (BHT), 1,4-benzoquinone, and 4-tert-butylcatechol.

27. A process for preparing a compound of Formula Ib, 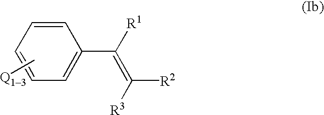
comprising:
reacting an aryl chloride of structural formula 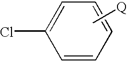
with an olefin of formula H(R¹)C=C(R²)(R³), wherein
Q is:
(1) halo, wherein halo is fluoro or chloro,
(2) (C₁—C₆)-alkyl,
(3) (C₁—C₆)-alkoxy,
(4) phenyl,
(5) oxo,
(6) hydroxy,
(7) NHCO(C₁—C₆)-alkyl,
(8) NR⁴R⁵, wherein R⁴ and R⁵ are independently hydrogen or (C₁—C₆)-alkyl,
(9) nitro,
(10) CO₂H,
(11) (C₁—C₄)- perfluoroalkyl,
(12) (C₁—C₄)- perfluoroalkoxy,
(13) cyano,
(14) SO₃H,
(15) CO₂(C₁—C₆)-alkyl,
(16) CO(C₁—C₆)-alkyl,
(17) CCl₃,
(18) CHO, or
(19) NR₃⁺ wherein R is hydrogen or (C₁—C₆)-alkyl; and R¹, R² and R³ are independently:
(1) hydrogen,
(2) (C₁—C₁₂)-alkyl,
(3) (C₂—C₁₂)-alkenyl,
(4) (C₁—C₁₂)-alkoxy,
(5) heteroaryl, wherein heteroaryl is defined as above,
(6) aryl, wherein aryl is defined as above,
(7) nitrile,
(8) silyl,
(9) NR⁴R⁵, wherein R⁴ and R⁵ are as defined above,
(10) NH(C₁—C₆)-alkyl-NR⁴R⁵, wherein R⁴ and R⁵ are as defined above,
(11) halo, wherein halo is fluoro, chloro, bromo, or iodo,
(12) (C₁—C₁₂)-alcohol,
(13) CO₂H,
(14) CO₂(C₁—C₁₂)-alkyl, or
(15) SR, wherein R is hydrogen or (C₁—C₆)-alkyl;
in the presence of a heterogeneous catalyst and a base at a temperature range of about 100° C. to about 180° C. in an aprotic solvent to generate the compound of Formula Ib.

28. The process of claim 27, wherein the Q is substituted in the position 4 of the phenyl ring.

29. The process of claim 28, wherein the heterogeneous catalyst is finely dispersed palladium on a solid support.

30. The process of claim 29, wherein the solid support is selected from the group consisting of carbon (Pd/C), silica, alumina, titania, and crystalline mesopourous zeolitic materials.

31. The process of claim 30, wherein the heterogeneous palladium catalyst is finely dispersed palladium without a solid support.

32. The process of claim 31, wherein the finely dispersed palladium is finely dispersed palladium metal (Pd Black) or finely dispersed palladium generated from homogeneous palladium acetate.

33. The process of claim 32, wherein the heterogeneous palladium catalyst is finely dispersed palladium (colloidal) stabilized by organic polymers.

34. The process of claim 33, wherein the aprotic solvent selected from the group consisting of N,N-dimethylacetamide (DMA), dioxane, N,N-dimethylformamide (DMF), N-methylpyrrolidinone (NMP), dioxane, ethylene glycol dimethyl ether (DME), diethoxymethane (DEM), tetrahydrofuran (THF), water, and a mixture thereof.

35. The process of claim 34, wherein the aprotic solvent is DMA, 1,4-dioxane, or a mixture thereof.

36. The process of claim 35, wherein the aprotic solvent is the mixture of DMA and 1,4-dioxane at a ratio of about 1:1 to about 1:5.

37. The process of claim 36, wherein the ratio of DMA to 1,4-dioxane is about 1:3.

38. The process of claim 37, wherein the base is selected from the group consisting of sodium acetate, potassium acetate, cesium acetate, triethylamine, trimethylamine, ethyldimethylamine, tri-n-propylamine, 1,4-diazabicyclio[2.2.2]octane, 1,8-diazabicyclo[5.4.0.]undec-7-ene (DBU), pyridine, lutidine, collidine, 4-dimethylaminomethyl-pyridine, sodium carbonate, potassium carbonate, cesium carbonate, sodium bicarbonate, potassium bicarbonate potassium, potassium methoxide, potassium ethoxide, sodium methoxide, sodium ethoxide, sodium tartrate, potassium tartrate, potassium bitartrate, sodium tartrate, and sodium bitartrate.

39. The process of claim 38, wherein the temperature range is about 140° C. to about 160° C.

40. The process of claim 27, which further comprises a radical scavenger selected from the group consisting of 4-methoxyphenol, bis(tert-butyl)hydroxy toluene (BHT), 1,4-benzoquinone, and 4-tert-butylcatechol.
